(19) United States
(12) Patent Application Publication
(10) Pub. No.: US 2023/0020956 A1
(43) Pub. Date: Jan. 19, 2023

(54) TWELVE-DIMENSIONAL BARBED SURGICAL THREAD

(71) Applicant: Eurothreads LLC, Hudson, WI (US)

(72) Inventors: Laurie K. Wall, Hudson, WI (US); Tamara Moore, Hudson, WI (US)

(21) Appl. No.: 17/864,889

(22) Filed: Jul. 14, 2022

Related U.S. Application Data
(60) Provisional application No. 63/221,512, filed on Jul. 14, 2021.
(60) Provisional application No. 63/235,234, filed on Aug. 20, 2021.

(51) Int. Cl. A61B 17/06; A61L 17/10

(52) U.S. Cl. CPC A61B 17/06166; A61L 17/10; A61B 2017/00792

(57) ABSTRACT

A twelve-dimensional barbed surgical thread that includes a central core and twelve first barbs. The central core has a circumference, a proximal end and a distal end that is opposite the proximal end. The twelve first barbs are positioned around the circumference of the central core so that the first barbs are radially adjacent to each other and obliquely extend from the central core.

### REFERENCE TO RELATED APPLICATION

This application claims priority to Provisional Applic. No. 63/221,512, filed on Jul. 14, 2021; and Provisional Applic. No. 63/235,234, filed on Aug. 20, 2021, the contents of which are incorporated herein by reference.

### FIELD OF THE INVENTION

The invention relates generally to thread for surgical applications. More particularly, the invention relates to twelve-dimensional barbed surgical thread.

### BACKGROUND OF THE INVENTION

A variety of aesthetic medical techniques have been developed to enhance the appearance of a person's skin and, in particular, the appearance of the person's face. These techniques range from cutting and tightening of the skin to injecting compositions into the skin such as to reduce wrinkles. Despite these advances, there is a continuing need to non-surgical techniques to improve a person's appearance.

### SUMMARY OF THE INVENTION

An embodiment of the invention is directed to a twelve-dimensional barbed surgical thread including a central code and twelve first barbs. The central core has a circumference, a proximal end and a distal end that is opposite the proximal end. The twelve first barbs are positioned around the circumference of the central core so that the first barbs are radially adjacent to each other and obliquely extend from the central core.

Another embodiment of the invention is directed to a method of using a twelve-dimensional barbed surgical. A twelve-dimensional barbed surgical thread is provided that includes a central core, twelve first barbs and twelve second barbs. The central core has a circumference, a proximal end and a distal end that is opposite the proximal end. The twelve first barbs are positioned around the circumference of the central core and obliquely extend from the central core. Each of the twelve first barbs has a distal end and a proximal end. The distal end of one of the first barbs is closer to the proximal end of the central core than the proximal end of the one of the second barbs. The twelve second barbs are positioned around the circumference of the central core and obliquely extend from the central core. The twelve second barbs are closer to the proximal end than the twelve first barbs. Each of the twelve second barbs has a distal end and a proximal end. The distal end of one of the second barbs is closer to the distal end of the central core than the proximal end of the one of the second barbs. The twelve-dimensional barbed surgical thread is inserted through a dermis and into a subcutaneous layer on a patient. The twelve first barbs engage the dermis to lift the dermis to a lifted position. The twelve second barbs engage the dermis to anchor the dermis in the lifted position.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a side view of a twelve-dimensional barbed surgical thread according to an embodiment of the invention.

FIG. 2 is a cross-sectional overview of the twelve-dimensional barbed surgical thread.

FIG. 3 is a photo in which the twelve-dimensional barbed surgical thread has been implanted on the right side of the person's face.

FIG. 4 is a photo in which the twelve-dimensional barbed surgical threads have been implanted on a left side of a person's buttocks.

### DETAILED DESCRIPTION OF THE INVENTION

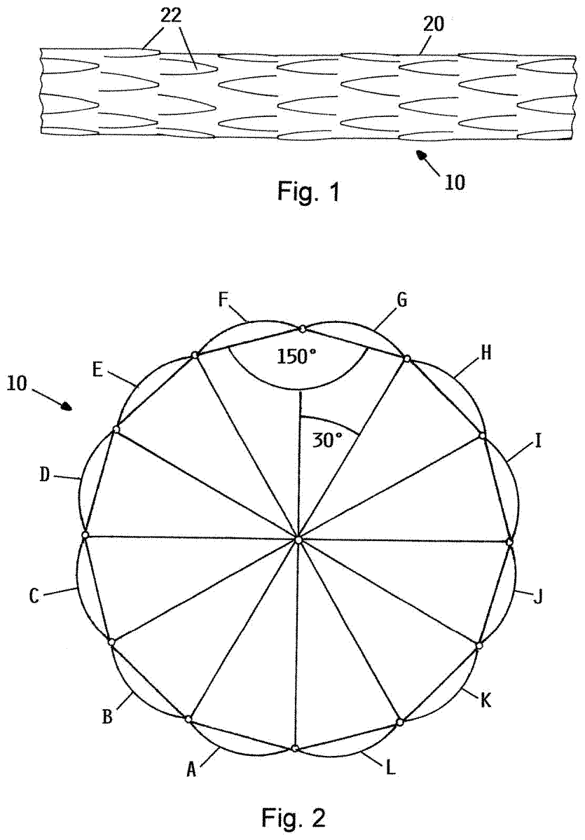

The invention is directed to a twelve-dimensional barbed surgical thread as illustrated at 10 in FIG. 1. The twelve-dimensional barbed surgical thread 10 is particularly suited for use in cosmetic enhancement procedures. The twelve-dimensional barbed surgical thread 10 includes a central core 20 from which a plurality of barbs 22 extend.

An advantage of the twelve-dimensional barbed surgical thread 10 over the prior art barbed surgical threads is that the twelve-dimensional barbed surgical thread 10 of this invention produces multiple effects once introduced into the dermis.

As compared to the six-dimensional barbed surgical thread and the eight-dimensional barbed surgical thread described in the recently filed patent applications owned by the assignee of this patent application, the twelve-dimensional barbed surgical thread provides superior performance when used in conjunction with heavier skin, thicker skin, tissue with greater elastin, collagen and/or volume loss.

In particular, the twelve-dimensional barbed surgical thread provides superior performance when used in conjunction with chest, buttocks and heavy facial tissue. For example, the twelve-dimensional barbed surgical thread is particularly suited for use in conjunction with at least one of chest lifts, buttocks lifts and face lifts where heavier tissue is involved hence the greater numbers of engagements into tissue of the twelve-dimensional barbed surgical thread would be of benefit due to the greater production of fibroblasts.

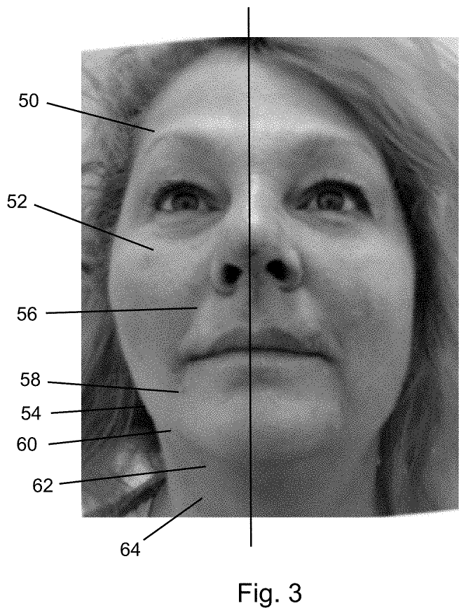

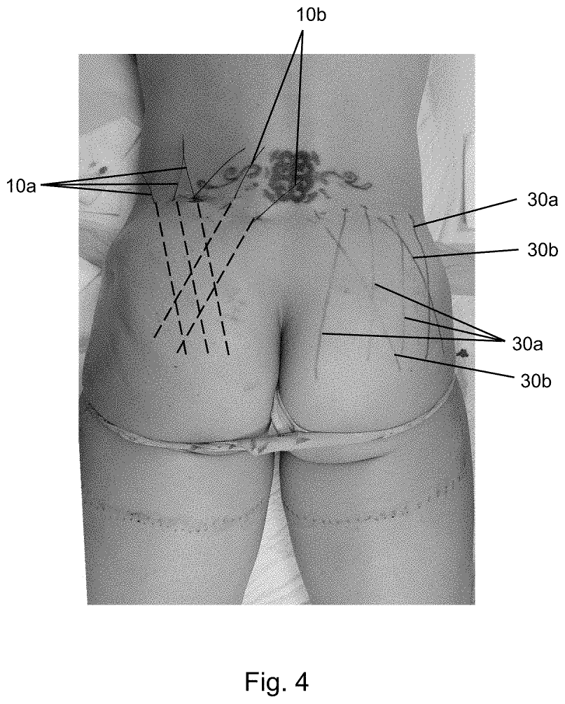

The twelve-dimensional barbed surgical thread is particularly beneficial to achieving results of a facial lift, which is described in detail below with respect to FIG. 3, and a buttock lift, which is described in detail below with respect to FIG. 4.

These effects fall into three major categories: (1) instant skin lifting through mechanical effects, (2) cellular renewal through collagen stimulation and neovascularization to improve skin texture, fine lines and elasticity by improving lymphatic flow, and (3) skin tightening by contracting fat tissue.

The twelve-dimensional barbed surgical thread 10 is a medical device that in certain embodiments consists of a monofilament surgical suture. The twelve-dimensional barbed surgical thread 10 may be fabricated from a variety of materials. In certain embodiments, the materials used to fabricate the twelve-dimensional barbed surgical thread is absorbable inside of a human body. A non-limiting example of a suitable material for fabricating the twelve-dimensional barbed surgical thread 10 is a polydioxanone (PDO)/polycaprolacton (PCL) polymer.

In certain embodiments, a cut is used to form each of the barbs 22 radially around the core 20. A variety of cutting techniques may be used. In certain embodiments, the twelve-dimensional barbed surgical thread 10 is fabricated by laser cutting into the surgical suture at an angle of about 30 degrees as illustrated in FIG. 2. The locations of the twelve cuts is indicated by letters A-L.

In certain embodiments, adjacent barbs 22 are offset in a direction extending between opposite ends of the twelve-dimensional surgical thread 10 such that a proximal end of one barb 22 is approximately aligned with a distal end of an adjacent barb 22. Using such a configuration causes the barbs 22 to be oriented in an angled configuration. The angled configuration can be oriented in a zigzag configuration as illustrated in FIG. 1. A person of skill in the art will appreciate that alternative configurations of the barbs 22 may be utilized to provide similar results when the twelve-dimensional surgical thread 10 is implanted in a patient.

Using such a configuration, the twelve barbs 22 are radially adjacent to each other when going around a circumference of the twelve-dimensional barbed surgical thread 10. While in certain embodiments, the barbs 22 are immediately radially adjacent to each other such as illustrated in FIG. 2, it is possible that there can be a spacing between adjacent barbs 22. As used herein, the spacing between adjacent barbs 22 is less than about 20 percent of the width of the widest portion of the barbs 22. In other embodiments, the spacing between adjacent barbs 22 is less than about 10 percent of the width of the widest portion of the barbs 22.

Using the preceding configuration enhances the potential of contacts between the barbs 22 after insertion into the dermis regardless of the orientation of the twelve-dimensional barbed surgical thread 10, which reduces the potential of the dermis sagging after insertion of the twelve-dimensional barbed surgical thread 10.

The cutting that is done to form the barb 22 is done at an angle to the surface of the central core 20. In certain configurations, the barbs 22 are oblique, notched protrusions formed into a surgical suture at an angle of between about 10 degrees and about 15 degrees. In other embodiments, the angle of the cut to form the barb 22 is about 12.5 degrees. Cutting in this manner causes a width of the barb 22 decreases when moving from the proximal end 40 to the distal end 42. In certain embodiments, the distal end 42 of the barb 22 is pointed.

In certain embodiments, a ratio of a length of the barb to a width of the barb is between about 2:1 and about 5:1. Forming the barbs 22 with the length to width ratio in this range provides the barbs 22 with sufficient strength for the barbs 22 to perform the desired functions during the insertion process and after insertion.

Adjacent barbs 22 may be offset in a direction moving from a distal end to a proximal end of the twelve-dimensional barbed surgical thread 10 as illustrated in FIG. 1. In one such embodiment, a distal end of one barb 22 is approximately aligned with a proximal end of an adjacent barb 22 around a circumference of the central core 20, as illustrated in FIG. 1. Additionally, the distal end of every other barb 22 is approximately aligned around a circumference of the central core 20, as illustrated in FIG. 1.

The barbs 22 have a proximal end and a distal end. The proximal end is where the barb 22 attaches to the central core 20. The distal end is opposite the proximal end.

The plurality of first barbs 22 are cut at intervals of about 30 degrees on twelve sides of the twelve-dimensional barbed surgical thread 10. In certain embodiments, the distance between adjacent barbs 22 is about 1.5 millimeters, which is closer than the conventional spacing of about 1.8 millimeters that is used in the prior art surgical thread that is used when performing facelifts.

The plurality of first barbs 22 proximate the distal end are oriented such that the distal end of each barb 22 is closer to the proximal end of the twelve-dimensional barbed surgical thread 10 than the proximal end of each barb 22. As described in more detail below, a primary function of the plurality of first barbs 22 is lifting.

In certain embodiments, the plurality of first barbs 22 occupy greater than about ½ of a length of the twelve-dimensional barbed surgical thread 10. In other embodiments, the plurality of first barbs 22 occupy about ⅔ of the length of the twelve-dimensional barbed surgical thread 10.

The twelve-dimensional barbed surgical thread 10 is typically inserted so that the plurality of first barbs 22 are facing upwardly. The configuration of the plurality of first barbs 22 on the twelve-dimensional barbed surgical thread 10 thereby allows for most of the twelve-dimensional barbed surgical thread 10 to assist in lifting of the tissue.

The twelve-dimensional barbed surgical thread 10 also includes a plurality of second barbs 24 proximate the proximal end 30. The twelve-dimensional barbed surgical thread 10 is typically inserted so that the plurality of second barbs 24 are facing downwards. As described in more detail below, a primary function of the plurality of second barbs 24 is anchoring.

The plurality of second barbs 24 are cut at intervals of about 30 degrees on twelve sides of the twelve-dimensional barbed surgical thread 10. In certain embodiments, the distance between adjacent barbs 24 is about 1.5 millimeters, which is closer than the conventional spacing of about 1.8 millimeters that is used in the prior art surgical thread that is used when performing facelifts.

Other than the orientation, the second barbs 24 may be shaped similarly to the first barbs 22. The plurality of second barbs 24 are oriented such that the distal end of each barb 24 is closer to the distal end 30 of the twelve-dimensional barbed surgical thread 10 than the distal end 40 of each barb 24.

In certain embodiments, the plurality of second barbs 24 occupies less than about ½ of the length of the twelve-dimensional barbed surgical thread 10. In other embodiments, the plurality of second barbs 24 occupies about ⅓ of the length of the twelve-dimensional barbed surgical thread 10.

In certain embodiments, there may be an intermediate region of the twelve-dimensional barbed surgical thread 10 that is intermediate the upwardly facing barbs 22 and the downwardly facing barbs 24 from which no barbs extend therefrom. This intermediate region on the twelve-dimensional barbed surgical thread 10 may have a length that is smaller than the length of the twelve-dimensional barbed surgical thread 10 over which the upwardly extending barbs 22 extend. The length of the intermediate region of the twelve-dimensional barbed surgical thread 10 may be smaller than the length of the twelve-dimensional barbed surgical thread 10 over which the downwardly extending barbs 24 extend.

Unlike the prior art two-dimensional, three-dimensional and four-dimensional barbed surgical thread that limited the ability to engage tissue, the twelve-dimensional barbed surgical thread 10 of this invention significantly improves the viability and uniformity of tissue engagement thus the sustainability of the desired result.

Additionally, upon insertion of the twelve-dimensional barbed surgical thread 10 into the dermis, the dermis sustains minor injuries. These minor injuries engages the body's natural healing process and stimulate the skin cells to produce collagen and blood vessels, which improves skin microcirculation and lymphatic flow.

It has been found that in areas of the body that do not have bone structure beneath, such as the fatty area of the cheek, having more points of tissue attachment provides a greater degree of lift, greater points of injury and a larger amount of fibroblasting, which thereby results in greater collagen production.

The twelve-dimensional barbed surgical 10 is inserted through a dermis and into a subcutaneous layer of the patient. In certain embodiments, a cannula is used to insert the twelve-dimensional barbed surgical thread 10 into the dermis . In other embodiments, the cannula is a rounded or L-tipped surgical steel cannula.

The twelve-dimensional barbed surgical thread 10 is not attached to the surgical cannula and is introduced into the soft tissue through the tip of the cannula. Once the cannula reaches the desired end site, the cannula is rotated about 180 degrees mechanically engaging the twelve-dimensional barbed surgical thread 10 at the distal (lower) end.

The cannula is then removed leaving only the twelve-dimensional barbed surgical thread 10 in the dermis. Approximating the tissue up the twelve-dimensional barbed surgical thread 10 (lifting the tissue onto each barb) allows for each of the barbs 22 to become securely engaged in the dermis. Once the desired result is achieved, the twelve-dimensional barbed surgical thread 10 is anchored at the proximal (upper) end. Any excess portion of the twelve-dimensional barbed surgical thread 10 is cut off and discarded.

The injection of twelve-dimensional barbed surgical threads 10 is indicated for soft tissue augmentation where the insertion of surgical sutures is appropriate. The twelve-dimensional barbed surgical threads 10 are used to lift, contour and volumize the skin. The implantation of the twelve-dimensional barbed surgical threads 10 is indicated for subcutaneous (intradermal and hypodermal) implantation.

In operation, once the area to be treated is defined, and an appropriate examination is completed, the patient is seated. The treatment area should be prepped by cleansing and removing the topical anesthetic. If topical anesthetic is to be used, it is applied liberally to the treatment areas.

The appropriate thread packages required for the treatment area are opened and removed from the package. The thread is attached in accordance with the manufacturer's instructions. Proper use of the product(s) should minimize the chances of dislodging or breaking while injecting the thread.

Correct insertion technique is important to the success of the treatment in achieving the desired results. The needle or cannula should be inserted into the treatment site with the tip ending up at an appropriate depth within the skin. The twelve-dimensional barbed surgical thread 10 should then be released using a slow, steady withdrawal of the needle/cannula. Overcorrection, which is more threads than suggested or required, is generally not needed and is to be avoided.

Once the first thread is appropriately inserted, another thread is inserted into the next adjacent location, and the process is repeated. Care should be taken to adequately assess the entire area to be treated with the correct number of threads to ensure even and symmetrical distribution of the product.

Once the injection is completed, the treated areas should be gently massaged, setting the threads per instruction. More vigorous massage may result in additional swelling, bruising or dislodgement of the thread.

Unlike the prior art two-dimensional, three-dimensional and four-dimensional barbed surgical threads that are limited in their ability to engage tissue due to barbs being cut respectively on only 2, 3 or 4 sides of the thread. This greatly limits the number of points of engagement into soft tissue. It is this engagement that is necessary to achieve a sustainable lift.

With twelve-dimensional barbed surgical threads 10, the presence of additional barbs 22 more than double the points of tissue engagement of the four-dimensional barbed surgical thread. Regardless of how the twelve-dimensional barbed surgical thread 10 is inserted into the subcutaneous layer, the additional barbs 22 allows the twelve-dimensional barbed surgical thread 10 to grab onto additional dermis resulting in a better lift.

The twelve-dimensional barbed surgical thread that is fabricated from PDO alone or in combination with PCL is slowly absorbed after implantation into the patient. In certain embodiments, the twelve-dimensional barbed surgical thread is resorbed over a time period of between about 180 days and about 240 days. After this time period, the twelve-dimensional barbed surgical thread 10 is substantially absorbed into the surrounding tissue. As used herein, substantially absorbed means that there are only minimal traces remaining of the twelve-dimensional barbed surgical thread 10

After implantation, the proximal ends of the twelve-dimensional barbed surgical threads 10 are trimmed so that the entirety of the remaining twelve-dimensional barbed surgical threads are recessed below the surface of the person's skin.

### EXAMPLE 1

In the following example, the performance of the twelve-dimensional barbed surgical thread according to this invention is evaluated. The subject on which the procedure was performed is a Caucasian female.

The twelve-dimensional barbed surgical threads have been implanted on the right side of the person's face, which is illustrated in FIG. 3. When comparing the ride side of the person's face to the left side of the person's face, there is a significant lift at the brow on the right side of the person's face. A corner of the right eye is lifted as compared to the left eye. The volume at the temple is increased on the right side of the person's face as compared to the left side of the person's face.

A volume at the right temple 50 is increased as compared to the left temple. There is a greater accentuation of the zygomatic arch 52 on the right side of the person's face as compared to the left side of the person's face. There is a thinning on the right side of the person's face as compared to the left side of the person's face.

Additionally, there is enhanced contouring and highlighting of the mandible 54 on the right side of the person's face as compared to the left side of the person's face. Nasolabial folds 56, marionette lines 58, jowls 60, submentum 62 and neck lines 64 are minimized on the right side of the person's face as compared to the left side of the person's face.

### EXAMPLE 2

In another embodiment of the invention, the twelve-dimensional surgical threads may be used to perform a buttocks lift. For example, FIG. 4 illustrates the twelve-dimensional surgical threads 10 implanted in a left side of a person's buttocks.

Lines 30a , 30b drawn on a right side of the person's buttocks generally indicate the direction in which the twelve-dimensional surgical threads 10 will be implanted. These lines include a first plurality of lines 30a that are oriented in a downward and inward direction and a second plurality of line 30b that are oriented in a downward and outward direction. As used herein, downward is with reference to a direction extending from a person's head to a person's feet. As used herein, inward means away from a side of the person. As used herein, outward means towards the side of the person.

In certain embodiments, a first plurality of the twelve-dimensional surgical threads 10a are implanted in a generally parallel orientation in a downward and inward direction.

While the first plurality of the twelve-dimensional surgical threads 10a are indicated as being implanted in a generally parallel orientation, there can be some deviation in the direction at which each of the first plurality of the twelve-dimensional surgical threads 10a are implanted.

The number of the twelve-dimensional surgical threads 10a in the first plurality may vary but is typically between about 2 and 10. In certain embodiments, there are 4 of twelve-dimensional surgical threads 10a in the first plurality.

It is also possible for a second plurality of the twelve-dimensional surgical threads 10b to be implanted in a generally parallel orientation in a downward and outward direction.

While the second plurality of the twelve-dimensional surgical threads 10b are indicated as being implanted in a generally parallel orientation, there can be some deviation in the direction at which each of the second plurality of the twelve-dimensional surgical threads 10b are implanted.

The number of the twelve-dimensional surgical threads 10b in the second plurality may vary but is typically between 2 and 10. In certain embodiments, there are 2 of twelve-dimensional surgical threads 10b in the second plurality.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A twelve-dimensional barbed surgical thread comprising:
a central core having a circumference, a proximal end and a distal end that is opposite the proximal end; and
twelve first barbs positioned around the circumference of the central core so that the first barbs are radially adjacent to each other and obliquely extend from the central core.

2. The twelve-dimensional barbed surgical thread of claim 1, and further comprising:
twelve second barbs positioned around the circumference of the central core, wherein the twelve second barbs are closer to the proximal end than the twelve first barbs, wherein each of the twelve second barbs has a distal end and a proximal end, wherein the distal end of one of the second barbs is closer to the distal end of the central core than the proximal end of the one of the second barbs, wherein each of the twelve first barbs has a distal end and a proximal end and wherein the distal end of one of the first barbs is closer to the proximal end of the central core than the proximal end of the one of the second barbs.

3. The twelve-dimensional barbed surgical thread of claim 2, wherein the twelve first barbs occupy about ⅔ of a length of the twelve-dimensional barbed surgical thread and wherein the twelve second barbs occupy about ⅓ of the length of the twelve-dimensional barbed surgical thread.

4. The twelve-dimensional barbed surgical thread of claim 1, wherein each of the twelve first barbs has a proximal end and a distal end and wherein adjacent barbs in the twelve first barbs are offset in a direction extending between the proximal end and the distal end of the central core so that the distal end of one of the twelve first barbs is approximately aligned with the proximal end of an adjacent one of the twelve first barbs around a circumference of the central core.

5. The twelve-dimensional barbed surgical thread of claim 1, wherein each of the twelve first barbs has a proximal end and a distal end and wherein every other of the twelve first barbs is approximately aligned around a circumference of the central core.

6. The twelve-dimensional barbed surgical thread of claim 1, wherein each of the first barbs occupies about 30 degrees of a circumference of the central core and wherein a cut is formed at an angle of between about 10 degrees and about 15 degrees with respect to a surface of the central core to form each of the first barbs.

7. The twelve-dimensional barbed surgical thread of claim 1, wherein the twelve-dimensional barbed surgical thread comprises a plurality of rows of the twelve first barbs and wherein a spacing between the rows of the twelve first barbs is less than about a length of one of the twelve first barbs in a direction that extends between the proximal end and the distal end of the central core.

8. The twelve-dimensional barbed surgical thread of claim 1, wherein the first barbs have a length to width ratio of between about 2:1 and about 5:1.

9. The twelve-dimensional barbed surgical thread of claim 1, wherein the twelve-dimensional thread is fabricated from polydioxanone/polycaprolactone polymer.

10. A method of using a twelve-dimensional barbed surgical comprising:
providing a twelve-dimensional barbed surgical thread comprising a central core, twelve first barbs and twelve second barbs, wherein the central core has a circumference, a proximal end and a distal end that is opposite the proximal end, wherein the twelve first barbs are positioned around the circumference of the central core and obliquely extend from the central core, wherein each of the twelve first barbs has a distal end and a proximal end, wherein the distal end of one of the first barbs is closer to the proximal end of the central core than the proximal end of the one of the second barbs, wherein the twelve second barbs are positioned around the circumference of the central core and obliquely extend from the central core, wherein the twelve second barbs are closer to the proximal end than the twelve first barbs, wherein each of the twelve second barbs has a distal end and a proximal end and wherein the distal end of one of the second barbs is closer to the distal end of the central core than the proximal end of the one of the second barbs; and
inserting the twelve-dimensional barbed surgical thread through a dermis and into a subcutaneous layer on a patient, wherein the twelve first barbs engage the dermis to lift the dermis to a lifted position and wherein the twelve second barbs engage the dermis to anchor the dermis in the lifted position.

11. The method of claim 10, wherein the twelve first barbs occupy about ⅔ of a length of the twelve-dimensional barbed surgical thread and wherein the twelve second barbs occupy about ⅓ of the length of the twelve-dimensional barbed surgical thread.

12. The method of claim 10, wherein each of the twelve first barbs has a proximal end and a distal end, wherein adjacent barbs in the twelve first barbs are offset in a direction extending between the proximal end and the distal end of the central core so that the distal end of one of the twelve first barbs is approximately aligned with the proximal end of an adjacent one of the twelve first barbs around a circumference of the central core, wherein each of the twelve first barbs has a proximal end and a distal end and wherein every other of the twelve first barbs is approximately aligned around a circumference of the central core.

13. The method of claim 10, wherein each of the first barbs occupies about 30 degrees of a circumference of the central core and wherein a cut is formed at an angle of between about 10 degrees and about 15 degrees with respect to a surface of the central core to form each of the first barbs.

14. The method of claim 10, wherein the twelve-dimensional barbed surgical thread comprises a plurality of rows of the twelve first barbs and wherein a spacing between the rows of the twelve first barbs is less than about a length of one of the twelve first barbs in a direction that extends between the proximal end and the distal end of the central core.

15. The method of claim 10, wherein the twelve-dimensional barbed surgical thread is inserted through the dermis into the subcutaneous layer using a cannula.

16. The method of claim 10, wherein insertion of the twelve-dimensional barbed surgical thread causes cellular renewal through collagen stimulation and neovascularization to improve skin texture by improving lymphatic flow.

17. The method of claim 10, wherein insertion of the twelve-dimensional barbed surgical thread into the subcutaneous layer causes skin tightening caused by contracting fat tissue.

18. The method of claim 10, wherein the insertion of the twelve-dimensional barbed surgical thread is in conjunction with at least one of a chest lift, buttocks lift and face lifts where heavier tissue is involved hence the greater numbers of engagements into tissue provided by the first barbs and the second barbs would be of benefit due to the greater production of fibroblasts.

19. The method of claim 10, wherein the insertion of the twelve-dimensional barbed surgical thread is in conjunction with at least one of heavier skin, thicker skin, tissue with greater elastin, collagen and or volume loss.

20. The method of claim 10, wherein the insertion of the twelve-dimensional barbed surgical thread provides enhance contact of the first barbs and the second barbs with the dermis in which the twelve-dimensional barbed surgical thread is inserted as compared to surgical thread having fewer than twelve dimensional barbs.
